(19) United States
(12) Patent Application Publication
(10) Pub. No.: US 2021/0228761 A1
(43) Pub. Date: Jul. 29, 2021

(54) Clean Air Device and Pass Box Including Ultraviolet Light Emitting Device

(71) Applicant: Hitachi Industrial Equipment Systems Co., Ltd., Tokyo (JP)

(72) Inventors: Takeshi KANEKO, Tainai (JP); Takeshi MATSUMURA, Tainai (JP)

(21) Appl. No.: 17/112,436

(22) Filed: Dec. 4, 2020

(30) Foreign Application Priority Data
Jan. 29, 2020 (JP) 2020-012369

(51) Int. Cl. A61L 9/20; H01L 25/075; A61L 2/10; F24F 8/22

(52) U.S. Cl. CPC A61L 9/20; H01L 25/0753; A61L 2209/12; F24F 8/22; A61L 2/10

(57) ABSTRACT

In a clean air device or a pass box that includes an ultraviolet light emitting device using a semiconductor chip, separately from a semiconductor chip that emits an ultraviolet ray, a semiconductor chip or an indicating lamp that emits visible light is disposed to visualize a strength of an ultraviolet intensity.

### BACKGROUND OF THE INVENTION

### 1. Field of the Invention

The present invention relates to a device that requires sterilization, specifically to a device such as a safety cabinet and an isolator that prevent a hazard which may occur during handling of cells, microorganisms, pathogens, or the like, a device such as a clean cabinet that maintains the cleanness of a work environment, and a device such as a pass box that prevents external contamination when a material is brought into a clean room, in industrial fields such as a regenerative medicine field where cells are cultured or a medical and pharmaceutical field where pathogens are handled and genes are manipulated.

### 2. Description of the Related Art

When cells or microorganisms are handled in research of pathogens or the like, in regenerative medicine, or the like, an isolator or a safety cabinet is used. When it is known in advance that pathogens or the like are not contained, a clean cabinet is used for the purpose of maintaining cleanliness. In addition, a pass box is used when a material is brought into a clean room.

For the purpose of sterilizing a surface of an article handled in the devices, a sterilization lamp may be used in addition to wiping with alcohol or the like. A low-pressure mercury lamp is mainly used as the sterilization lamp, and due to containing a very small amount of mercury, the low-pressure mercury lamp may lead to environmental contamination and the disposal method may be complicated. As an alternative product, a semiconductor chip that emits ultraviolet rays may be used.

JP 2016-111364 A discloses one example of an ultraviolet light emitting device using a semiconductor chip.

When the low-pressure mercury lamp is used as the sterilization lamp, since a very small amount of visible light in a range of 400 to 500 nm is contained, light can be seen in bluish purple. However, when the semiconductor chip which emits an ultraviolet ray is used as an alternative product, the peak of a wavelength in a deep ultraviolet (UV-C) range, which has a high sterilization effect, can be created, and light having a wavelength other than the range is not output. Therefore, the light of the sterilization lamp is invisible, and it cannot be determined whether or not the sterilization lamp is turned on and whether or not ultraviolet rays are emitted.

JP 2016-111364 A describes the structure of the ultraviolet light emitting device using the semiconductor chip; however, the installation of the ultraviolet light emitting device in an apparatus such as a clean air device, or emission light visualizing means is not taken into consideration.

### SUMMARY OF THE INVENTION

An object of the present invention is to provide a clean air device or a pass box that uses a semiconductor chip to perform sterilization, the semiconductor chip emitting an ultraviolet ray, in which the intensity of the ultraviolet ray is visualized, so that an article can be installed in a region having high sterilization power.

In order to solve the above problems, as an example of “a clean air device and a pass box including an ultraviolet light emitting device” of the present invention, there is provided a clean air device or a pass box that includes an ultraviolet light emitting device using a semiconductor chip, in which separately from a semiconductor chip that emits an ultraviolet ray, a semiconductor chip or an indicating lamp that emits visible light is disposed to visualize a strength of an ultraviolet intensity.

According to the present invention, in the clean air device or the pass box that uses the semiconductor chip to perform sterilization, the semiconductor chip emitting an ultraviolet ray, the intensity of the ultraviolet ray is visualized, so that an article can be installed in a region having high sterilization power.

Tasks, configurations, and effects other than those described above become apparent from the description of the following embodiments.

### BRIEF DESCRIPTION OF THE DRAWINGS

FIG. 1A is a view seen from a side surface and illustrating one example of an ultraviolet light emitting device of a first embodiment;

FIG. 1B is a view seen from a bottom surface side to which semiconductor chips are attached, and illustrating one example of the ultraviolet light emitting device of the first embodiment;

FIG. 2 is a view illustrating one example of a pass box including the ultraviolet light emitting device of the first embodiment;

FIG. 3 is an image showing one example where the strength of the ultraviolet intensity is visualized in light and dark in the pass box including the ultraviolet light emitting device of the first embodiment;

FIG. 4 is a view illustrating a modification example of the ultraviolet light emitting device of the first embodiment;

FIG. 5 is an image showing one example where the strength of the ultraviolet intensity is visualized in warm and cold tones in a second embodiment;

FIG. 6 is a view illustrating one example where a sterilization surface is visualized with a stainless steel hairline material in a pass box including an ultraviolet light emitting device of a third embodiment;

FIG. 7 is a view illustrating an example of visualization of light when the sterilization surface is made of the stainless steel hairline material in the pass box including the ultraviolet light emitting device of the third embodiment;

FIG. 8 is a view illustrating one example of a pass box including an ultraviolet light emitting device of a fourth embodiment; and

FIG. 9 is a view illustrating a bottom surface of a work chamber in the pass box including the ultraviolet light emitting device of the fourth embodiment.

### DETAILED DESCRIPTION OF THE PREFERRED EMBODIMENTS

Embodiments of the present invention will be described with reference to the drawings. Incidentally, in the drawings for describing the embodiments, the same names and reference signs will be assigned to the same components whenever possible, and repeated descriptions thereof will be omitted.

### First Embodiment

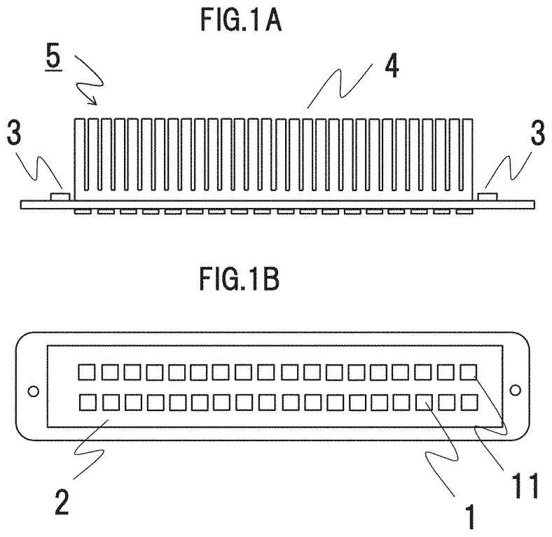

FIG. 1A is a view seen from a side surface and illustrating one example of an ultraviolet light emitting device of a first embodiment. FIG. 1B is a view seen from a bottom surface side to which semiconductor chips are attached.

An ultraviolet light emitting device 5 includes a semiconductor chip 1 that emits ultraviolet rays, a semiconductor chip 11 that emits visible light, a substrate 2 on which the semiconductor chip 1 and the semiconductor chip are disposed, a connector 3 to which a wiring for power supply is connected, and a heat radiation fin 4, and is modularized. On one surface of the substrate 2, a plurality of the semiconductor chips 1 that emit ultraviolet rays are disposed in a row, and a plurality of the semiconductor chips that emit visible light are disposed in a row adjacently along the semiconductor chips 1. The heat radiation fin 4 that radiates heat from the semiconductor chips 1 and 11 is provided on an opposite surface of the substrate 2. The semiconductor chip 1 that emits ultraviolet rays outputs ultraviolet rays, for example, UV-C waves (deep ultraviolet rays) having a wavelength in a range of 100 to 280 nm which is effective for sterilization. The semiconductor chip 11 that emits visible light outputs visible light having, for example, a wavelength of 400 nm to 800 nm.

Incidentally, in FIGS. 1A and 1B, the semiconductor chip and the semiconductor chip 11 are configured as an integrated module to facilitate the attachment of the ultraviolet light emitting device 5; however, the semiconductor chip 1 and the semiconductor chip 11 may be separated from each other. In addition, the disposition or number of the semiconductor chips can be appropriately changed.

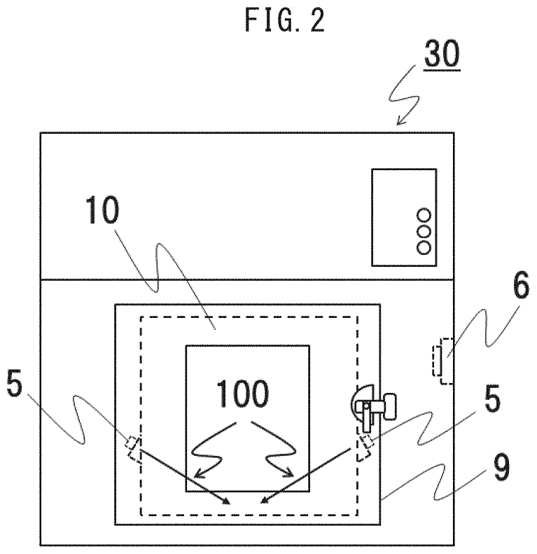

FIG. 2 illustrates one example of a pass box including the ultraviolet light emitting device 5 of the first embodiment. The pass box is equipment that is installed on an inlet side or outlet side of a clean room to allow an article to be put into and out of the clean room. Doors are provided on a clean room side and a front chamber side of the pass box, and are configured to not be opened at the same time such that dust or the like is prevented from entering the clean room.

In FIG. 2, the ultraviolet light emitting device 5 and an ultraviolet light emitting device controller 6 are attached to a pass box 30. The ultraviolet light emitting devices 5 illustrated in FIGS. 1A and 1B are attached to both side surfaces of a work chamber 10 to irradiate a bottom surface of the work chamber with ultraviolet rays 100, an article being placed on the bottom surface. Reference sign 9 denotes the door of the pass box, and the pass box 30 includes the doors on a front side and a back side of the drawing.

An operation of the pass box including the ultraviolet light emitting device of FIG. 2 will be described. When electricity is supplied from the connector 3 to the semiconductor chip 1 that emits ultraviolet rays, the semiconductor chip 1 emits ultraviolet rays to irradiate the article with the ultraviolet rays 100, the article being disposed on the bottom surface of the work chamber 10. At the same time, since the semiconductor chips having the same irradiation angle are used, visible light is emitted from the semiconductor chip 11 which is disposed adjacent to the semiconductor chip 1 to emit visible light, so that the strength of the light can be visually observed.

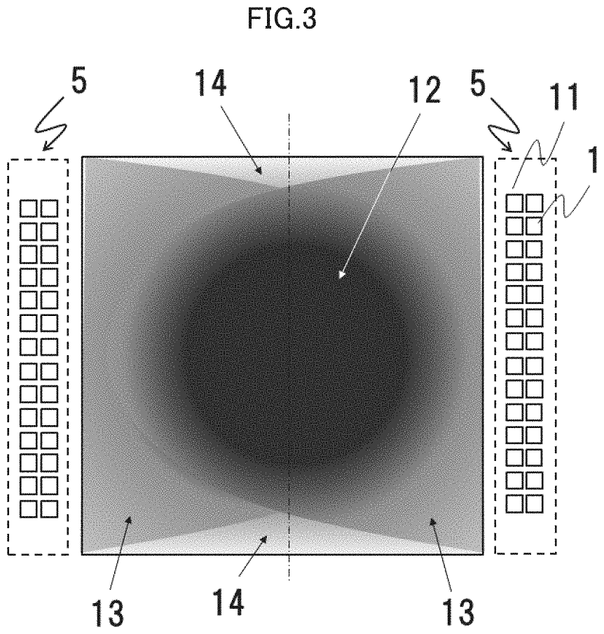

FIG. 3 shows one example of the visual effect, and the light intensity of a region 12 which has a strong sterilization effect and in which ultraviolet rays of the ultraviolet light emitting devices 5 installed on the facing surfaces intersect each other is strong, and a region 13 and a region 14 therearound, the region 13 having a medium sterilization effect and the region 14 having a weak sterilization effect, can be visually confirmed. Then, an article requiring sterilization is placed in the region 12 where the ultraviolet intensity is strong, and an article such as a resin material of which the ultraviolet ray-induced deterioration is desired to be prevented is placed in the region 13 or 14 where the ultraviolet intensity is weak, so that the ease of use is improved.

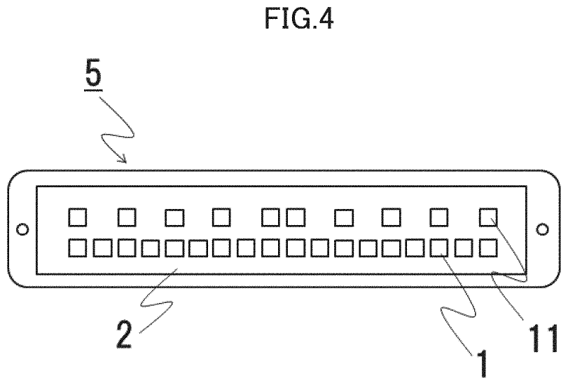

FIG. 4 illustrates a modification example of the ultraviolet light emitting device. In the modification example, the number of the semiconductor chips 11 for visualization which emits visible light is decreased. The number of the semiconductor chips can be appropriately adjusted according to the strength of light formed by light emission of the semiconductor chip 11 for visualization.

Incidentally, instead of the semiconductor chip 11 for visualization which outputs visible light, an indicating lamp such as a neon lamp may be used. In addition, in FIG. 2, the ultraviolet light emitting devices 5 are disposed on wall surfaces on both sides of the work chamber, but may be disposed on an upper side of the work chamber, such as a ceiling of the work chamber, to irradiate the bottom surface of the work chamber with ultraviolet rays.

According to the present embodiment, since the semiconductor chips 11 that emit visible light are arranged adjacent to the semiconductor chips 1 that emit ultraviolet rays, visible light having an intensity distribution equivalent to that of ultraviolet rays emitted from the semiconductor chips 1 is emitted. Therefore, the intensity of the ultraviolet rays, namely, the strength of the sterilization effect can be visually observed, and an article can be installed in a region having high sterilization power. In addition, the high straightness property of the semiconductor chip can be used to irradiate a required region with ultraviolet rays of the required intensity, which leads to cost reduction and power saving.

In addition, when the amount of ultraviolet rays is measured in advance and the number of the semiconductor chips which emit visible light is determined or the semiconductor chips are disposed such that the intensity of the visible light is high only in a region irradiated with the required ultraviolet rays or more, zoning can be performed according to the strength of light. In addition, on the contrary, a region where ultraviolet rays are weak is irradiated with specific visible light, so that an unusable region can be zoned in tones.

### Second Embodiment

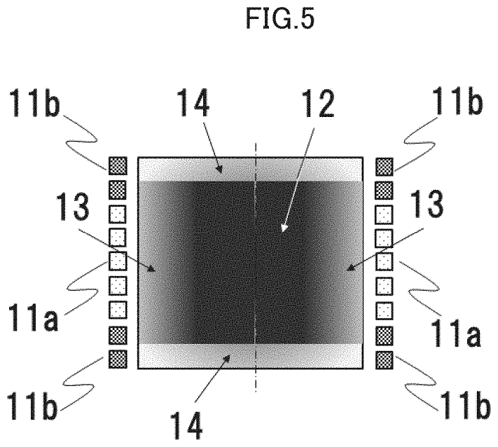

FIG. 5 shows one example where the strength of the ultraviolet intensity is visualized in warm and cold tones in a second embodiment. Two or more types of the semiconductor chips 11 for visualization having different wavelengths from that of the semiconductor chip 1 (not illustrated) for emission of ultraviolet rays are arranged to create tones. In the drawing, for example, reference sign 11a denotes semiconductor chips that are disposed in central portions in a vertical direction to emit a warm color such as red, and reference sign 11b denotes semiconductor chips that are disposed on both sides in the vertical direction to emit a cold color such as blue. In the drawing illustrating a bottom surface of a work chamber, reference sign 12 denotes a region having a strong ultraviolet intensity and a strong sterilization effect, and the region is displayed in a warm color. Reference sign 13 denotes a region having a medium ultraviolet intensity and a medium sterilization effect, and the region is displayed in a weak warm color. Reference sign 14 denotes a region having a weak ultraviolet intensity and a weak sterilization effect, and the region is displayed in a cold color. When a region having a high ultraviolet intensity is displayed in a warm color such as red and a region having a low ultraviolet intensity is displayed in a cool color such as blue, it is easy to distinguish the regions from each other.

In the drawing, the semiconductor chips that emit two types of visible light are provided; however, more types of semiconductor chips may be disposed.

According to the present embodiment, the strength of the ultraviolet intensity can be visually observed due to a change in warm and cold tone, so that an article can be installed in a region having the required sterilization power.

### Third Embodiment

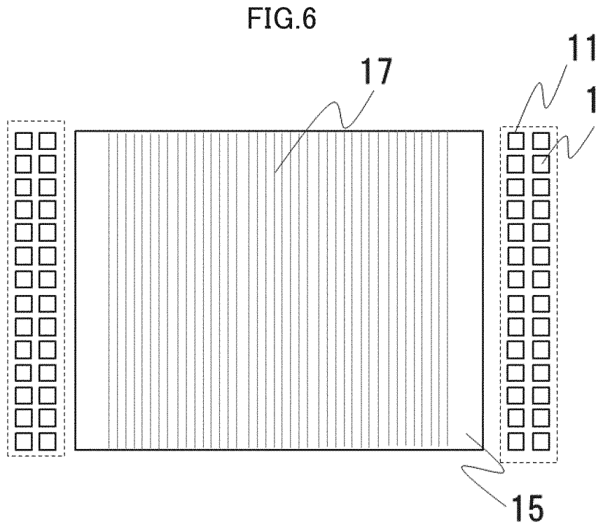

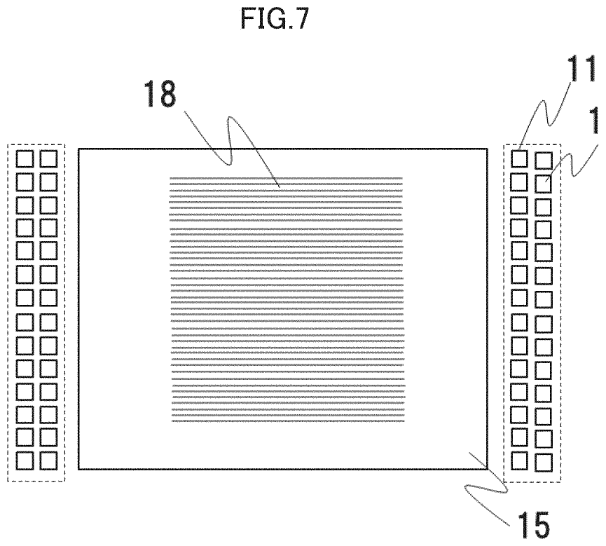

FIG. 6 illustrates one example of a pass box including an ultraviolet light emitting device of a third embodiment. FIG. 6 illustrates an example where a stainless steel hairline material is used as the material of a bottom surface 15 that is a sterilization surface of the pass box. The stainless steel hairline material is produced by forming a plurality of parallel fine grooves in a surface of a stainless steel plate. A stainless steel hairline direction 17 is set to be parallel to the arrangement of the semiconductor chips 11 for visualization which have an irradiation angle of 120° or less and output visible light having high straightness. Accordingly, as illustrated in FIG. 7, light is reflected by the fine grooves of the hairlines, the color of the visible light of the semiconductor chips 11 for visualization can be seen in a stripe pattern 18, and an ultraviolet irradiation region can be seen at a glance. Since the stripe pattern 18 cannot be seen in a region which cannot be effectively irradiated with ultraviolet rays, it can be seen at a glance that the region has no sterilization effect.

According to the present embodiment, the strength of the ultraviolet intensity can be seen in light and dark tones, and the irradiation region can also be seen due to the stripe pattern formed in the bottom surface of the work chamber.

### Fourth Embodiment

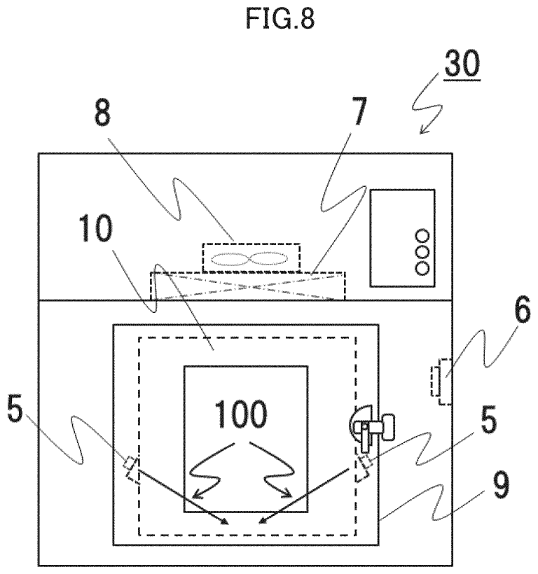

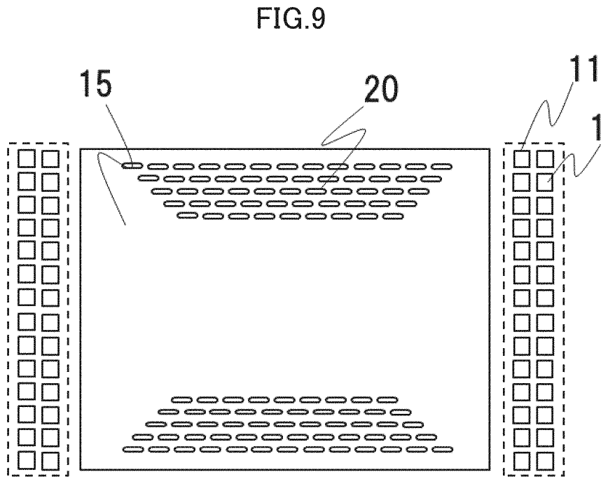

FIG. 8 illustrates one example of a pass box including an ultraviolet light emitting device of a fourth embodiment. In addition, FIG. 9 illustrates a bottom surface of a work chamber of the pass box of the fourth embodiment. The fourth embodiment is an example of the pass box having an improved dust removal efficiency.

In order to maintain cleanness inside the pass box, a HEPA filter 7 and a fan 8 are built-in in the pass box of FIG. 2 to form the pass box 30 of FIG. 8. In a state where a door 9 of the pass box is closed, an article being input and output through the door 9, clean air is blown into the work chamber, and the clean air circulates, so that the cleanliness of the work chamber 10 can be secured. Here, the HEPA filter is an abbreviation for a high efficiency particulate air filter.

In order to obtain a sterilization effect by ultraviolet irradiation, it is necessary to keep an irradiated surface as clean as possible and keep the irradiated surface without dirt. If there is dirt, ultraviolet rays may be blocked and an effective sterilization effect may not be obtained. Therefore, as illustrated in FIG. 9, circulation slits 20 are provided on door 9 sides at the front and rear of the pass box in a bottom surface 15 of the work chamber such that dust generated from an article is removed from the work chamber 10 in the shortest time. Then, the passing wind speed is kept at approximately 5 m/s to improve the air circulation efficiency, so that the cleanliness of the work chamber is maintained. Incidentally, the slits are alternately disposed in a staggered manner to not overlap each other in a forward and rearward direction, so that dust can be effectively removed. In addition, since the slits are not provided in a central portion of the bottom surface and are provided only near the doors at the front and rear, the wind speed can be increased, and dust can be effectively removed. In addition, dust generated when the door 9 of the pass box is opened and an article is put into the work chamber 10 is suppressed from leaking outside the pass box, and the contamination of the clean room outside the pass box is suppressed.

According to the present embodiment, when an article is put from outside into the work chamber which is a sterilization surface, dust adhering to the article is collected by the HEPA filter in the pass box without leaking outside the pass box, and thus the contamination of the work chamber is suppressed and the sterilization effect by ultraviolet rays of the ultraviolet light emitting device can be improved as much as possible.

In each of the embodiments, the pass box has been described as an example of the present invention; however, the present invention can be used in any clean air device which requires sterilization, such as a safety cabinet, an isolator, or a clean cabinet.

What is claimed is:

1. A clean air device that includes an ultraviolet light emitting device using a semiconductor chip,
wherein separately from a semiconductor chip that emits an ultraviolet ray, a semiconductor chip or an indicating lamp that emits visible light is disposed to visualize a strength of an ultraviolet intensity.

2. The clean air device according to claim 1,
wherein the semiconductor chip that emits the ultraviolet ray and the semiconductor chip that emits the visible light are disposed on the same substrate to be modularized.

3. The clean air device according to claim 1,
wherein the strength of the ultraviolet intensity is visualized in light and dark tones or in warm and cold tones.

4. The clean air device according to claim 1,
wherein the clean air device is a safety cabinet, an isolator, or a clean cabinet.

5. A pass box that includes an ultraviolet light emitting device using a semiconductor chip,
wherein separately from a semiconductor chip that emits an ultraviolet ray, a semiconductor chip or an indicating lamp that emits visible light is disposed to visualize a strength of an ultraviolet intensity.

6. The pass box according to claim 5,
wherein the semiconductor chip that emits the ultraviolet ray and the semiconductor chip that emits the visible light are disposed on the same substrate to be modularized.

7. The pass box according to claim 5,
wherein the semiconductor chips that emit the ultraviolet rays and the semiconductor chips that emit the visible light are disposed on wall surfaces on both sides of a work chamber to irradiate a bottom surface of the work chamber with the ultraviolet rays and the visible light.

8. The pass box according to claim 5,
wherein the strength of the ultraviolet intensity is visualized in light and dark tones or in warm and cold tones.

9. The pass box according to claim 5,
wherein a stainless steel hairline material is used as a material of a surface requiring sterilization, and
a plurality of the semiconductor chips that emit the visible light are linearly disposed in parallel to a hairline direction of the stainless steel hairline material.

10. The pass box according to claim 5,
wherein a fan that circulates air and a HEPA filter that removes dust are built-in, and
a plurality of circulation slits are arranged in a bottom surface of a work chamber to be close and parallel to an opening and closing door.
